(19) United States
(12) Patent Application Publication
(10) Pub. No.: US 2024/0238201 A1
(43) Pub. Date: Jul. 18, 2024

(54) LIPOSOME CARRIER-BASED MULTI-TARGET COMPLEX, DRUG DELIVERY PLATFORM CONTAINING THE SAME, AND APPLICATION THEREOF

(71) Applicant: HANGZHOU XINGAO BIOTECHNOLOGY CO., LTD., Hangzhou (CN)

(72) Inventors: Xiangshi TAN, Hangzhou (CN); Daofeng XIANG, Hangzhou (CN); Yingxuan TAN, Hangzhou (CN)

(21) Appl. No.: 18/624,880

(22) Filed: Apr. 2, 2024

Related U.S. Application Data
(63) Continuation of application No. PCT/CN2022/103417, filed on Jul. 11, 2022.

(30) Foreign Application Priority Data
Aug. 5, 2021 (CN) 202110896817.1

(51) Int. Cl. A61K 9/127; A61K 31/517; A61K 31/7084; A61K 47/64; A61K 47/69; A61P 35/00; A61P 35/04

(52) U.S. Cl. CPC A61K 9/127; A61K 31/517; A61K 31/7084; A61K 47/646; A61K 47/6913; A61P 35/00; A61P 35/04

(57) ABSTRACT

A liposome carrier-based multi-target complex, which includes a stimulator of interferon genes (STING) agonist, an immune checkpoint inhibitor, an ectonucleotide pyrophosphatase/phosphodiesterase (ENPP1) inhibitor, and a liposome. The immune checkpoint inhibitor is configured to target at least two different immune checkpoints or antigenic epitopes. A weight ratio of the STING agonist to the ENPP1 inhibitor is 10:2.5-10, excluding 10:10, and a weight ratio of the STING agonist to the immune checkpoint inhibitor is 10:2.5-100, excluding 10:100. This application also provides a drug delivery platform and applications of the multi-target complex.

### REFERENCE TO AN ELECTRONIC SEQUENCE LISTING

The contents of the electronic sequence listing (SequenceListing.xml; Size: 3,279 bytes; and Date of Creation: Apr. 2, 2024) is herein incorporated by reference in its entirety.

### CROSS-REFERENCE TO RELATED APPLICATIONS

This application is a continuation of International Patent Application No. PCT/CN2022/103417, filed on Jul. 11, 2022, which claims the benefit of priority from Chinese Patent Application No. 202110896817.1, filed on Aug. 5, 2021. The content of the aforementioned application, including any intervening amendments thereto, is incorporated herein by reference in its entirety.

### TECHNICAL FIELD

This application relates to biomedical technology, and more specifically to a liposome carrier-based multi-target complex, a drug delivery platform containing the same, and an application thereof.

### BACKGROUND

Tumors seriously endanger human life and health. Immunotherapy, distinguished from the conventional tumor treatment, has obtained a considerable development in recent years. Immunotherapy refers to a therapy that uses a person's own immune system to attack tumor cells and thus control the development and metastasis of the tumor. However, it is difficult for the immune system to find tumors before the canceration, and the immune function may be suppressed through immune checkpoints during the development of the tumors, resulting in immune escape. Therefore, two key points in the employment of the immune system to fight against tumors are: (1) to enhance the natural immunity through immune pathway activators/agonists, and (2) to disrupt the suppression of immune checkpoints on immune cells and prevent the immune escape. The cancer immunotherapy is predominated by an immune checkpoint inhibitor (i.e., monoclonal antibody drugs) and cellular immunotherapy.

Stimulator of interferon genes (STING), also known as mediator of IRF3 activation (MITA), human transmembrane protein 173 (TMEM173), endoplasmic reticulum interferon stimulator (ERIS), nuclear envelope transmembrane protein 23 (NET23), or membrane tetraspanner (MPYS), is an endoplasmic reticulum (ER) receptor protein. Activation of STING can enhance the ability of the innate immune system to fight tumors or infections. Microbial and viral DNA in infected mammalian cells can induce potent endogenous immune responses by stimulating the interferon secretion. Cyclic GMP-AMP (cGAMP), an agonist of the innate immune STING pathway, has been verified to be effective in the tumor treatment. It can activate the immune cells such as dendritic cells through immune activation or strengthening of the innate immune pathway, thereby inducing the activation of cytotoxic T cells to kill tumor cells.

The immune response of the ER receptor proteins (e.g., STING) to cytoplasmic DNA is essential. It has been shown that the cyclic GMP-AMP synthetase (cGAS) can be activated upon binding to DNA to endogenously catalyze the synthesis of the cyclic dinucleotide cGAMP. The cGAMP serves as a second messenger to stimulate the response to interferon IFN-I via STING, and mediate the activation of TBK1 and IRF-3, thereby triggering the transcription of the type I interferon IFN-β gene. STING is an ER-associated transmembrane protein, and there is a hydrolytic enzyme ectonucleotide pyrophosphatase/phosphodiesterase 1 (ENPP1) on the ER. ENPP1 exists as a monomer with a molecular weight of 115 kD in a reduced state, and as a dimer with a molecular weight of 230 kD in a non-reduced state. ENPP1 is a transmembrane protein located in the cell membrane or ER, and can be secreted extracellularly as a soluble N-terminally cleaved monomer. In addition to the phosphodiester bond in natural nucleotides, the ENPP1 can also catalyze the hydrolysis of the cGAMP. It has been demonstrated in mice that the knockout of the ENPP1 gene can significantly increase the half-life of cGAMP. The cGAMP may leave the virus-infected cell or cancer cell to transfer to other host cells in a specific way, and then activate the STING signaling pathway in the host cell to induce the expression of type I interferon. Since the cGAMP is prone to degradation by the free extracellular ENPP1, it is required to prevent the STING agonist cGAMP from being hydrolyzed by ENPP1 to maintain efficacy within an extended period. Hence, highly-efficient ENPP1 inhibitors play an important role in improving the efficacy of tumor immunotherapy.

Nanobody is a kind of single-domain antibodies, in which the entire light chain and the constant region 1 of the heavy chain (CH1) are absent, and exhibits unique properties of stable structure, small size, excellent solubility, superior tolerance to adverse environments, and easy humanization. However, different nanoantibodies have their own specificity, and the application in drugs is still in the clinical research stage.

As a drug carrier, the nanoliposome has a promising prospect in the drug half-life extension, drug efficacy enhancement, and targeted drug delivery. However, it is still challenging to prepare targeted liposomes with good stability, high encapsulation rate and superior cellular uptake. To further improve the targetability and utilization of drugs, immune-targeted liposomes such as monoclonal antibodies have been developed, which can target immune cells and tumor microenvironments. Nevertheless, monoclonal antibodies struggle with large molecular weight, high preparation cost, and potential immune response, limiting the clinical application and promotion.

### SUMMARY

An objective of the present disclosure is to provide a liposome carrier-based multi-target complex, a drug delivery platform containing the same, and an application thereof to overcome the defect in the prior art that there is a lack of a liposome carrier with excellent stability, high encapsulation rate, and superior efficacy and targeted drug delivery ability. The multi-target complex of the present disclosure is co-loaded with a stimulator of interferon genes (STING) agonist, an ectonucleotide pyrophosphatase/phosphodiesterase (ENPP1) inhibitor, and an immune checkpoint inhibitor, and can rapidly and precisely target the tumor microenvironment. Moreover, by combining an active immune activator with a blocker antibody that can block the tumor immune escape, the anti-tumor activity is greatly enhanced.

It has been found that when an outer surface of the liposome is linked to the antibody, the liposome can act as an engager between the tumor cell and the immune cell within the tumor microenvironment, promoting the interaction between the tumor cell and the immune cell. Therefore, an attempt has been made to combine the liposome with the immune checkpoint inhibitor, the immune pathway agonist and the tumor-targeting agent, and the results demonstrate that the combined liposome can effectively relieve the immunosuppression in the tumor microenvironment and block the immune escape, thus enhancing the targeting effect and the inhibitory effect of the immune checkpoint inhibitor, and further synergistically enhancing the antitumor effect. However, the inhibition of the immune checkpoint and activation of immune pathways may lead to immune reactions, such as cytokine storm. To balance the interactions between various inhibitors and receptors, the present disclosure introduces various inhibitors and obtains a liposome carrier-based multi-target complex that can effectively improve anti-tumor effects without generating an immune storm through a considerable number of complicated experiments. To further optimize the performance of the multi-target complex, an antibody, which has small molecular weight, good tissue penetration ability, high specificity, high affinity and weak immunogenicity, and can avoid the complement binding reaction at the Fc segment, is selected as a component of the multi-target complex. Through screening of the existing antibodies or antigen-binding fragments thereof, it is found that the nanoantibody has stronger epitope recognition and binding ability, and small size, which enables the high-density and firm binding to the solid phase carrier to capture trace antigens, thereby effectively improving the binding activity of the multi-target complex.

Technical solutions of the present disclosure are as follows.

In a first aspect, this application provides a liposome carrier-based multi-target complex, comprising:
a stimulator of interferon genes (STING) agonist;
an immune checkpoint inhibitor;
an ectonucleotide pyrophosphatase/phosphodiesterase (ENPP1) inhibitor; and
a liposome;
wherein the liposome is a closed unilamellar vesicle having a concentric lipid bilayer membrane and a hydrophilic core; the STING agonist is located in the hydrophilic core, and the ENPP1 inhibitor is located in a hydrophobic interlayer of the concentric lipid bilayer membrane; and the immune checkpoint inhibitor is linked to an outer surface of the liposome;
the immune checkpoint inhibitor is configured to target at least two different immune checkpoint or antigenic epitopes; and
a weight ratio of the STING agonist to the ENPP1 inhibitor is 10:2.5-10, excluding 10:10; and a weight ratio of the STING agonist to the immune checkpoint inhibitor is 10:2.5-100, excluding 10:100.

In some embodiments, the weight ratio of the STING agonist to the ENPP1 inhibitor is 10:2.5-5; and the weight ratio of the STING agonist to the immune checkpoint inhibitor is 10:2.5-50.

In some embodiments, the weight ratio of the STING agonist to the immune checkpoint inhibitor is 10:5-50, preferably, 10:5-10.

In some embodiments, the STING agonist is an agonist of a cyclic GMP-AMP synthetase (cGAS)-STING-cyclic GMP-AMP (cGAMP)-interferon regulatory factor 3 (IRF3) immune pathway.

In some embodiments, the agonist is a cyclic dinucleotide or a derivative thereof, such as cyclic dinucleotide 2′3′-cGAMP (c-AMP-GMP), c-di-AMP, c-di-GMP, c-di-IMP, c-GMP-IMP and substituted derivatives thereof.

In some embodiments, the cyclic dinucleotide metal derivative comprises a transition metal compound, such as zinc and manganese cyclic dinucleotide compound.

In the present disclosure, STING is a specific protein name, which, if not stated, is consistent with most of the disclosed literature and the NCBI database and the European Gene Database. The GENE name of STING is TMEM173, and its GENE ID is 340061. Other disclosed designations of STING include transmembrane protein 173, ERIS, MITA, MPYS, NET23, SAVI, hMITA, and hSTING.

In the present disclosure, unless otherwise specified, the cyclic dinucleotide cGAMP (i.e., 2′3′-cGAMP) all refer to C₂₀H₂₄N₁₀O₁₃P₂.

In some embodiments, the immune checkpoint inhibitor is an antibody against an immune checkpoint or an antigen-binding fragment thereof.

In some embodiments, the immune checkpoint inhibitor is an antibody against an immune checkpoint.

In some embodiments, the antibody is a nanobody (VHH), a monoclonal antibody or a VH-Fc recombinant antibody thereof.

In some embodiments, the immune checkpoint is selected from the group consisting of PD-1/PD-L1, CD47, VEGF, PVRIG, TIGIT, NKG2A, LILRB4, LILRB2, LAG-3, OX40, CTLA-4, TIM-3, VISTA, ILDR2, KIR, MHCII, GITR, 4-1BB and a combination thereof.

In some embodiments, the immune checkpoint is PD-1/PD-L1 or CD47.

In the present disclosure, the ENPP1 inhibitor is a humanized ENPP1 inhibitor.

In some embodiments, the ENPP1 inhibitor is a 4(3H)-quinazolinone derivative.

In some embodiments, the ENPP1 inhibitor is selected from the group consisting of 2-[(4-methyl-(thiazolo[4,5-B]pyridinyl)-2-thio)methyl]-7-fluoro-quinazoline-4(3H)-one, 2-[(5-methoxy-(imidazo[4,5-B]pyridinyl)-2-thio)methyl]-7-fluoro-quinazoline-4(3H)-one and 2-[(5-chloro-(imidazo[4,5-B]pyridine)-2-thio)methyl]-7-methoxycarbonyl-quinazoline-4(3H)-one.

In the present disclosure, the antigenic epitope of the immune checkpoint may be a cell surface antigenic epitope, an intracellular antigenic epitope, or a secreted expressed antigenic epitope. At least two different immune checkpoint antigenic epitopes are synergistic with each other.

In some embodiments, raw materials of the liposome comprises a phospholipid and cholesterol; the phospholipid is selected from hydrogenated soybean phospholipid, dipalmitoyl phosphatidylcholine, dipalmitoyl phosphatidylglycerol, dipalmitoyl-phosphatidylethanolamine-polyethylene glycol 2000, dipalmitoyl phosphatidylethanolamine-polyethylene glycol 2000-maleoethanolamine, 1,2-distearoyl-SN-glycerol-3-phosphoethanolamine-N-maleimide-polyethylene glycol 2000 (DSPE-PEG2000-MAL) and a combination thereof, and a weight ratio of the phospholipid to the cholesterol is 18-25:5.

In some embodiments, the weight ratio of the phospholipid to the cholesterol is 19-21:5, such as 19:5, 20:5 and 21:5.

In some embodiments, the multi-target complex further comprises a tumor-targeting reagent, wherein the tumor-targeting reagent is bridged to the outer surface of the liposome through a chemical bond.

In some embodiments, the tumor-targeting reagent is a ligand targeting a receptor specifically expressed by a tumor cell.

In some embodiments, the tumor-targeting reagent is folic acid or integrin.

In the present disclosure, the tumor-targeting reagent can bind to a specific receptor of a tumor cell to inhibit aberrant signaling, such as cyclooxygenase 2 (COX-2), folic acid, or neovascularization.

In some embodiments, the liposome carrier-based multi-target complex is prepared through steps of:
(S1) subjecting raw materials of the liposome and the ENPP1 inhibitor to thin film hydration to prepare a loaded liposome, wherein the raw materials of the liposome comprise a phospholipid and cholesterol; and
(S2) subjecting the loaded liposome and the STING agonist to a first incubation under stirring, and adding the immune checkpoint inhibitor for a second incubation under stirring to obtain the liposome carrier-based multi-target complex;
wherein the first incubation is performed at 65-75° C. for 1.5-2.5 h, such as 70° C. for 2 h; and the second incubation is performed at 20-25° C. overnight in the dark.

In some embodiments, the loaded liposome is prepared from the raw materials of the liposome, the ENPP1 inhibitor and a tumor-targeting reagent through thin film hydration.

In some embodiments, a weight ratio of the STING agonist to the tumor-targeting reagent is 10:2.5-10, excluding 10:10.

In the present disclosure, the film hydration method is a conventional technology in the art. For example, raw materials of the liposome can be dried by vacuum rotary evaporation in a water bath to form a film, and then (NH₄)₂SO₄ is added for hydration to prepare a blank liposome. An immune agonist or a metal complex thereof, and an ENPP1 inhibitor are added to the blank liposome to form a co-loaded liposome. After that, the liposome is chemically bonded to link an immune checkpoint inhibitor (e.g., a monoclonal antibody (or a nano-antibody)), followed by removal of unencapsulated STING agonist or ENPP1 inhibitor, and unlinked antibody protein through dialysis and molecular sieving.

In s second aspect, this application provides a method for preparing the liposome carrier-based multi-target complex, comprising:
(S1) subjecting raw materials of the liposome and the ENPP1 inhibitor to thin film hydration to prepare a loaded liposome, wherein the raw materials of the liposome comprise a phospholipid and cholesterol;
wherein the raw materials of the liposome comprise a phospholipid and cholesterol; the phospholipid is selected from hydrogenated soybean phospholipid, dipalmitoyl phosphatidylcholine, dipalmitoyl phosphatidylglycerol, dipalmitoyl-phosphatidylethanolamine-polyethylene glycol 2000, dipalmitoyl phosphatidylethanolamine-polyethylene glycol 2000-maleoethanolamine (DSPE-PEG₂₀₀₀), 1,2-distearoyl-SN-glycerol-3-phosphoethanolamine-N-maleimide-polyethylene glycol 2000 (DSPE-PEG₂₀₀₀-MAL) and a combination thereof; and
(S2) subjecting the loaded liposome and the STING agonist to a first incubation under stirring, and adding the immune checkpoint inhibitor for a second incubation under stirring to obtain the liposome carrier-based multi-target complex;
wherein a weight ratio of the STING agonist to the ENPP1 inhibitor is 10:2.5-10, excluding 10:10, and a weight ratio of the STING agonist to the immune checkpoint inhibitor is 10:2.5-100, excluding 10:100; the first incubation is performed at 65-75° C. for 1.5-2.5 h, such as 70° C. for 2 h, and the second incubation is performed at 20-25° C. overnight in the dark.

In some embodiments, a weight ratio of the STING agonist to the ENPP1 inhibitor is 10:2.5-5, and a weight ratio of the STING agonist to the immune checkpoint inhibitor is 10:2.5-50.

In a third aspect, this application provides a drug delivery platform, comprising:
the aforementioned liposome carrier-based multi-target complex.

In a fourth aspect, this application provides a pharmaceutical composition, comprising:
the aforementioned liposome carrier-based multi-target complex; and
a pharmaceutically-acceptable carrier;
wherein an administration route of the pharmaceutical composition comprises injection, drip and oral administration;
the injection is intravenous injection, intramuscular injection or subcutaneous injection; and
the drip is intravenous drip or nasal drip.

In some embodiments, the pharmaceutical composition further comprising:
a therapeutic agent;
wherein the therapeutic agent comprises a peptide or an antibody targeting a receptor protein of a blood-brain barrier, or fragments thereof.

In some embodiments, the pharmaceutical composition further comprising:
a therapeutic agent;
wherein the therapeutic agent comprises a peptide or an antibody targeting a receptor protein of a blood-brain barrier, or fragments thereof.

In a fifth aspect, this application provides a method for treating a tumor or a tumor metastasis in a subject in need thereof, comprising:
administering a therapeutically effective amount of the aforementioned pharmaceutical composition to the subject;
wherein the tumor is a solid tumor; the solid tumor is selected from the group consisting of colorectal cancer, breast cancer, ovarian cancer, prostate cancer, pancreatic cancer, testicular cancer, lung cancer, nasopharyngeal cancer, esophageal cancer, renal cancer, glioma, melanoma, malignant lymphoma, head and neck cancer, thyroid cancer and osteogenic sarcoma; and the tumor metastasis is selected from the group consisting of lung metastasis, liver metastasis, lymphatic metastasis, brain metastasis and bone metastasis.

In the present disclosure, the multi-targeting complex of a carrier liposome can be connected to a plurality of targeting molecules according to the need for targeting, forming a multi-antibody-targeting drug delivery platform through the liposome co-loaded with the STING agonist and the ENPP1 inhibitor, co-loaded with the STING agonist and the antitumor drug, and co-loaded with the STING agonist, the ENPP1 inhibitor and the antitumor drug.

In a sixth aspect, this application provides a method for treating a viral inflammation associated with a coronavirus in a subject in need thereof, comprising:
administering a therapeutically effective amount of the aforementioned pharmaceutical composition 14 to the subject;
wherein the coronavirus is selected from the group consisting of 2019-novel coronavirus (2019-nCoV), influenza virus and human immunodeficiency virus (HIV); and the viral inflammation is selected from the group consisting of Corona Virus Disease 2019 (COVID-19), viral nephritis, viral encephalitis, viral enteritis and viral hepatitis.

In a seventh aspect, this application provides a method for treating a neurodegenerative disease in a subject in need thereof, comprising:
administering a therapeutically effective amount of the aforementioned pharmaceutical composition to the subject;
wherein neurodegenerative disease is selected from the group consisting of Alzheimer's disease, Parkinson's disease, amyotrophic lateral sclerosis, multiple sclerosis, ataxia telangiectasia, bovine spongiform encephalopathy, Creutzfeldt-Jakob disease, Huntington's chorea, spinocerebellar atrophy, spinal muscular atrophy, spastic paraplegia and myasthenia gravis.

In an eighth aspect, this application provides a method for treating a brain disease in a subject in need thereof, comprising:
administering a therapeutically effective amount of the aforementioned pharmaceutical composition to the subject;
wherein the brain disease is selected from the group consisting of ischemic cerebrovascular injury, craniocerebral injury, encephalitis, and brain tumor.

On the basis of common knowledge in the art, the above preferred conditions can be arbitrarily combined to obtain the preferred examples of the present invention.

The reagents and raw materials used herein are commercially available.

The beneficial effects of the present disclosure are presented as follows.

The multi-target complex of the carrier liposome of the present disclosure is co-loaded with a STING agonist, an ENPP1 inhibitor and an immune checkpoint inhibitor, which has a better anti-tumor effect than the complex loaded with a single STING agonist or a single ENPP1 inhibitor. Moreover, the addition of the tumor-targeting reagent (such as folic acid) further improves the anti-tumor activity of the multi-target complex. Therefore, the multi-target complex provided herein possesses a promising prospect for application in the design of anti-tumor drugs. In addition, the present disclosure can also be used to prepare drugs for the treatment of corresponding diseases by replacing the co-loaded components and immune checkpoint inhibitors connected to the surface of liposomes as needed, which has a high clinical application value.

The present disclosure integrates the effects of the natural immune agonist with the inhibitor thereof, the immune checkpoint inhibitor and antibodies thereof, and liposomes to obtain the multi-targeting complex, which avoids the rapid degradation of the immune agonist in vivo, rapidly and precisely targets the tumor microenvironment, and actively combines the active immune activator with the antibody of the blocking agent that prevents the immune escape. The preferred nanoantibody is easy for mass production and low cost, reducing the overall production cost and facilitating the promotion.

### DETAILED DESCRIPTION OF EMBODIMENTS

The present disclosure is further described below with reference to embodiments, but the present disclosure is not limited to the scope of the described embodiments. The specific conditions not indicated in the experimental methods in the following embodiments are selected in accordance with conventional methods and conditions, or with the descriptive literature.

### Example 1 Preparation of an Innate Immune Stimulator of Interferon Genes (STING) Agonist cGAMP and an (Ectonucleotide Pyrophosphatase/Phosphodiesterase) ENPP1 Inhibitor

(1) The STING immune agonist cyclic dinucleotide 2′,3′-cGAMP was synthesized as described in the literature (Pingwei Li, et al., Immunity, 2013, 39(6), 1019-1031) under an activation condition after binding DNA, which was prepared from cyclized cGMP-AMP dinucleotide synthase (cGAS) through catalyzed synthesis. The purity was analyzed by high performance liquid chromatography (HPLC) and mass spectrometry identification to be above 98%.

### (2) Preparation and Characterization of the ENPP1 Inhibitor

### (2-1) Preparation and Characterization of ENPP1-YZ1

Methyl 2-amino-4-fluorobenzoate (338 mg, 2 mmol) and chloroacetonitrile (127 μL, 2 mmol) were added to 4 M of HCl/dioxane (5 mL) and reacted at 97° C. overnight. After that, the reaction solution was cooled to the room temperature and slowly dropwise added with 20 mL of saturated sodium bicarbonate solution under an ice bath to precipitate a solid, followed by suction filtration and rinsing sequentially with water, ethanol and ether to obtain 239 mg of brown solid product, i.e., 2-(chloromethyl)-7-fluoroquinazoline-4(3H)-one (56% yield).

¹H NMR (400 MHz, DMSO) δ 12.69 (s, 1H), 8.19 (t, J=8.2 Hz, 1H), 7.48 (d, J=8.4 Hz, 1H), 7.41 (t, J=8.7, 1H), 4.53 (s, 2H).

¹³C NMR (101 MHz, DMSO) δ 167.49, 164.99, 161.27, 154.36, 150.99, 129.53, 129.42, 118.76, 116.37, 116.14, 113.08, 112.86, 43.51.

5-methoxy-2-mercaptoimidazo [4,5-b] pyridine (91 mg, 0.5 mmol), 2-(chloromethyl)-7-fluoroquinazoline-4(3H)-one (106 mg, 0.5 mmol) and sodium hydroxide (100 mg, 2.5 mmol) were dissolved in methanol (7 mL) and stirred at the room temperature overnight. After that, the organic solvent methanol was removed under reduced pressure to obtain a crude product. The crude product was purified using a 300-400-mesh silica gel column to obtain 122 mg of white solid product as ENPP1-YZ1, i.e., (2-[(5-methoxy-(imidazo[4,5-B] pyridinyl)-2-thio) methyl]-7-fluoroquinazoline-4(3H)-one) (68% yield).

¹H NMR (400 MHz, DMSO) δ 13.22 (s, 1H), 12.69 (s, 1H), 8.22-8.10 (m, 1H), 7.83 (s, 1H), 7.48-7.36 (m, 2H), 6.63 (d, J=8.6 Hz, 1H), 4.51 (s, 2H), 3.86 (s, 3H).

¹³C NMR (101 MHz, DMSO) δ 167.35, 164.84, 162.59, 161.10, 154.45, 150.11, 149.98, 147.43, 144.67, 129.54, 129.43, 126.20, 121.88, 118.44, 116.05, 115.82, 112.21, 111.99, 109.60, 54.52, 36.00.

ESI-MS m/z calcd for C₁₆H₁₂FN₅O₂S+358.0768, found 358.0762[M+H]⁺.

### (2-2) Preparation and Characterization of ENPP1-YZ2

4-Methyl-2-mercaptobenzothiazole (91 mg, 0.5 mmol), 2-(chloromethyl)-7-fluoroquinazoline-4(3H)-one (106 mg, 0.5 mmol) and sodium hydroxide (100 mg, 2.5 mmol) were dissolved in methanol (7 mL) and stirred at room temperature overnight. After that, the organic solvent methanol was removed under reduced pressure to obtain a crude product. The crude product was purified using a 0-400 mesh silica gel column to obtain 100 mg of white solid product as ENPP1-YZ2, i.e., 2-[(4-methyl-(thiazolo[4,5-B]pyridinyl)-2-thio)methyl]-7-fluoro quinazoline-4(3H)-one (56% yield).

¹H NMR (400 MHz, DMSO) δ 12.66 (s, 1H), 8.17 (dd, J=8.7, 2.4 Hz, 1H), 7.86-7.76 (m, 1H), 7.38 (ddd, J=11.2, 9.5, 2.5 Hz, 2H), 7.29-7.20 (m, 2H), 4.62 (s, 2H), 2.58 (s, 3H).

¹³C NMR (101 MHz, DMSO) δ 167.49, 164.99, 164.22, 161.02, 156.09, 151.93, 149.73, 149.60, 135.16, 131.33, 129.72, 129.61, 127.36, 125.28, 119.64, 118.14, 116.19, 115.96, 111.87, 111.65, 35.95, 18.23.

ESI-MS m/z calcd for C₁₇H₁₂FN₃OS₂⁺358.0479, found 358.0488[M+H]+.

### Example 2 Preparation of a Liposome Loaded with cGAMP and ENPP1-YZ1

450 mg of hydrogenated soybean phosphatidylcholine (HSPC), 150 mg of cholesterol CHOL, 120 mg of DPPE-PEG₂₀₀₀ and 5 mg of ENPP1-YZ1 were added into 50 mL of dichloromethane, and transferred to a 1 L eggplant-shaped flask for vacuum spinning dry to form a film. Then, continue to spinning dry for more than 3 h to remove the residual trace organic solvent dichloromethane. 60 mL of 250 mM ammonium sulfate solution was added for hydration at 60° C. The hydrated liposome was extruded from a liposome extruder and filtered through a 100 nm polycarbonate microporous filtration membrane to form a homogeneous unicellular liposome. The homogeneous liposome was loaded into a 3500 Da dialysis bag and fully dialyzed to remove the free small-molecule inhibitor ENPP1-YZ1 and ammonium sulfate, where the buffer for dialysis was 500 mL of 5% dextrose solution; the dialysis was carried out three times; and the dialysate was changed every 6 h. 55 mg of cGAMP was added to the liposome solution and incubated under slight stirring with a magnetic stirrer in an oil bath at 70° C. for 2 h. The incubated liposome was loaded into a 3500 Da dialysis bag and fully dialyzed to remove free cGAMP, where the buffer for dialysis was 500 mL of 5% dextrose solution; the dialysis was carried out three times; and the dialysate was changed every 6 h. The dialyzed liposome was added with 10 wt. % alginate and freeze-dried to obtain the cGAMP/ENPP1-YZ1 loaded liposome (complex I) lyophilized powder, which was stored at −20° C.

The encapsulation rate determination of the cGAMP/ENPP1-YZ1 loaded liposome was performed as follows. A certain amount of cGAMP/ENPP1-YZ1 loaded liposome lyophilized powder was added with 700 μL of emulsion breaker (methanol:isopropanol=7:3, V/V) and 300 μL of water, oscillated for uniform mixing, and centrifuged at 10,000 rpm for 1 min to obtain a supernatant. The supernatant was subjected to a high-performance liquid chromatography (HPLC) test to determine the concentration of the drug through the concentration-peak area standard curve, and the encapsulation rate of the ENPP1-YZ1 was calculated to be 97%, and the encapsulation rate of cGAMP was calculated to be 95%.

### Example 3 Preparation of Anti-PD-L1/Anti-CD47 Nanobodies (VHHs)

### (1) Construction of Anti-PD-L1/Anti-CD47 Nanobody Plasmids

The sequence of the nano-antibody plasmid was designed referring to the literature (Broos, K., et al., Oncotarget, 2017.8(26):41932-41946; Sockolosky, J. T., et al., Proc Natl Acad Sci USA, 2016.113(19):E2646-54.). It was verified that the nanobodies encoded by this sequence had strong interaction with murine-derived PD-L1/or CD47.

The above two genes were murine-derived nanobodies. PET-22b(+) was used as a vector carrying Amp+ resistance. The end of the protein sequence was labeled with 6His-tag to assist purification. The expression system was Escherichia coli (E. coli). Plasmids were synthesized by Universal Biosystems Ltd. The nanoantibodies were all efficiently expressed with E. coli.

(a) Protein sequence of the anti-PD-L1 nanobody
(SEQ ID NO: 1):
QVQLQESGGGLVQTGGSLRLSCAASGSTVSSSMMAWWRQTPGNQRE
LVALVASGNNTNYVDSVKGRFTVSRDNAKNTMYLQMNSLKPEDTAVYYC
RILSVNGIWYWGQGTQVTVSS
(b) Protein sequence of the anti-CD47 nanobody
(SEQ ID NO: 2):
QVQLVESGGGLVEPGGSLRLSCAASGIIFKINDMGWYRQAPGKRREW
VAASTGGDEAIYRDSVKDRFTISRDAKNSVFLQMNSLKPEDTAVYYCTA
VISTDRDGTEWRRYWGQGTQVTVSS

### (2) Purification of Nanobody Protein

Bacteria suspension was added with P1 buffer (HEPES 50 mM, pH 7.5, 150 mM NaCl) in 1 mL/g, stirred for bacteriolysis, broken with an ultrasonic cell wall breaker, centrifuged, and separated to collect the supernatant.

The Ni-NTA affinity column was equilibrated with P1 buffer and centrifuged to obtain the supernatant. The supernatant was rinsed with P2 buffer (HEPES 50 mM, pH 7.5, 150 mM NaCl, 20 mM Im) containing 20 mM imidazole (Im) to remove heteroproteins, and rinsed with P1 buffer and P3 buffer (HEPES 50 mM, pH 7.5, 150 mM NaCl, 200 mM Im) gradient to remove target proteins. The target protein eluate was dialyzed to remove imidazole, subjected to fractional purity determination using a 15% (m/v) SDS-PAGE and mass spectrometry identification, and stored at −80° C. for use.

### (3) Identification of Nanobodies

The concentration of the nanobody protein solution r was about 1 mg/mL. The mass spectral molecular weights of the anti-PD-L1 VHH and the anti-CD47 VHH was 13.83 kD and 14.05 kD, respectively, as detected by MALDI-TOF-MS analysis. The purity was identified as higher than 95% for both of the anti-PD-L1 VHH and the anti-CD47 VHH using a 15% (m/v) SDS-PAGE.

### (4) Removal Endotoxin of Nanobody Proteins

After equilibrating the Ni-NTA column with PBS, the dialyzed nanobody protein solution was loaded onto the column. The Ni-NTA column was rinsed with PBS solution containing 0.1% (v/v) TritonX-114 (40 times the column volume) to remove endotoxin. Finally, the target protein was eluted with a gradient of LPS-free PBS solution containing imidazole. Then the collected protein was dialyzed to remove the imidazole, and concentrated to about 1.5 mg/mL using a 10-kd ultrafiltration tube (Millipore). After that, the endotoxin content in the nanobody protein was confirmed to be <2 IU/mg using an endotoxin kit, and the nanobody protein was stored at −80° C. for use.

### Example 4 Preparation of Anti-CD47-Targeted cGAMP/ENPP1-YZ1 Loaded Liposome

450 mg of HSPC, 150 mg of cholesterol CHOL, 120 mg of DPPE-PEG₂₀₀₀30 mg of DPPE-PEG₂₀₀₀-MAL and 5 mg of ENPP1-YZ1 were added into 50 mL of dichloromethane, and transferred to a 1 L eggplant-shaped flask for vacuum spinning dry to form a film. Then, continue to spinning dry for more than 3 h to remove the residual trace organic solvent dichloromethane. 60 mL of 250 mM ammonium sulfate solution was added for hydration at 60° C. The hydrated liposome was extruded from a liposome extruder and filtered through a 100 nm polycarbonate microporous filtration membrane to form a homogeneous unicellular liposome. The homogeneous liposome was loaded into a 3500 Da dialysis bag and fully dialyzed to remove the free small-molecule inhibitor ENPP1-YZ1 and ammonium sulfate, where the buffer for dialysis was 500 mL of 5% dextrose solution; the dialysis was carried out three times; and the dialysate was changed every 6 h. 55 mg of cGAMP was added to the liposome solution and incubated under slight stirring with a magnetic stirrer in an oil bath at 70° C. for 2 h. After the incubation, the anti-CD47 VHH prepared in Example 3 that had been sulfhydrylated (total mass of phospholipid: mass of anti-CD47 nanoantibody=1 mg:20 g) and the reducing agent (sodium hydrosulfite solution, with a final concentration of 1 mM) were added for incubation overnight at the room temperature in the dark. After that, the liposome loaded with the anti-CD47 nanoantibody was loaded into a 30 kD dialysis bag and fully dialyzed to remove free anti-CD47 nanoantibodies and cGAMP, where the buffer for dialysis was 500 mL of 5% dextrose solution; the dialysis was carried out three times; and the dialysate was changed every 6 h. The dialyzed liposome was added with 10 wt. % alginate and freeze-dried to obtain complex II lyophilized powder, which was stored at −20° C.

The encapsulation rate determination of the complex II was performed as follows. A certain amount of the complex II was added with 700 μL of emulsion breaker (methanol:isopropanol=7:3, V/V) and 300 μL of water, oscillated for uniform mixing, and centrifuged at 10,000 rpm for 1 min to obtain a supernatant. The supernatant was subjected to HPLC tests to determine the concentration of the drug through the concentration-peak area standard curve, and the encapsulation rate of the ENPP1-YZ1 was calculated to be 96%, and the encapsulation rate of cGAMP was calculated to be 94%.

The protein ligation of complex II was determined as follows. 300 μL of a liposome solution was added with 0.4 mL of methanol for vortex shaking for 30 s, added with 0.2 mL of dichloromethane for vortex shaking for 30 s, and added with 0.1 mL dd H₂O for vortex shaking for 30 s. Then the mixture solution was centrifuged at 9000 g for 1 min, and the upper layer was removed to obtain the organic dichloromethane layer. Then 0.3 mL methanol was added for vortex shaking for 30 s, and centrifuged at 9000 g for 2 min, and the supernatant was carefully removed to obtain a white precipitate. The residual solvent was dried with N₂, and add 200 μL of HEPES (20 mM HEPES,140 mM NaCl, 2% SDS) to solubilize the protein. The anti-CD47 protein ligation of complex II was determined to be 95% according to the test method of BCA kit (Omni-Rapid™ Rapid Protein Quantification Kit, ZJ103).

### Example 5 Preparation of Anti-PD-L1/Anti-CD47-Targeted cGAMP/ENPP1-YZ1 Loaded Liposomes

450 mg of HSPC, 150 mg of cholesterol CHOL, 120 mg of DPPE-PEG₂₀₀₀60 mg of DPPE-PEG₂₀₀₀-MAL and 5 mg of ENPP1-YZ1 were added into 50 mL of dichloromethane, and transferred to a 1 L eggplant-shaped flask for vacuum spinning dry to form a film. Then, continue to spinning dry for more than 3 h to remove the residual trace organic solvent dichloromethane. 60 mL of 250 mM ammonium sulfate solution was added for hydration at 60° C. The hydrated liposome was extruded from a liposome extruder and filtered through a 100 nm polycarbonate microporous filtration membrane to form a homogeneous unicellular liposome. The homogeneous liposome was loaded into a 3500 Da dialysis bag and fully dialyzed to remove the free small-molecule inhibitor ENPP1-YZ1 and ammonium sulfate, where the buffer for dialysis was 500 mL of 5% dextrose solution; the dialysis was carried out three times; and the dialysate was changed every 6 h. 55 mg of cGAMP was added to the liposome solution and incubated under slight stirring with a magnetic stirrer in an oil bath at 70° C. for 2 h. After the incubation, the anti-PD-L1 VHH and anti-CD47 VHH prepared in Example 3 that had been sulfhydrylated (total weight of phospholipid:weight of anti-PD-L1 VHH:weight of anti-CD47 VHH=1 mg:20 μg: 20 μg) and the reducing agent (sodium hydrosulfite solution, with a final concentration of 1 mM) were added for incubation overnight at the room temperature in the dark. After that, the liposome loaded with the anti-PD-L1 VHH and the anti-CD47 VHH was loaded into a 30 kD dialysis bag and fully dialyzed to remove free anti-PD-L1 VHH, the anti-CD47 VHH and cGAMP, where the buffer for dialysis was 500 mL of 5% dextrose solution; the dialysis was carried out three times; and the dialysate was changed every 6 h. The dialyzed liposome was added with 10 wt. % alginate and freeze-dried to obtain complex III lyophilized powder, which was stored at −20° C.

The encapsulation rate determination of the complex III was performed as follows. A certain amount of the complex III was added with 700 μL of emulsion breaker (methanol:isopropanol=7:3, V/V) and 300 μL of water, oscillated for uniform mixing, and centrifuged at 10,000 rpm for 1 min to obtain a supernatant. The supernatant was subjected to HPLC analysis to determine the concentration of the drug through the concentration-peak area standard curve, and the encapsulation rate of the ENPP1-YZ1 was calculated to be 94%, and the encapsulation rate of cGAMP was calculated to be 95%.

The protein ligation of complex III was determined as follows. 300 μL of a liposome solution was added with 0.4 mL of methanol for vortex shaking for 30 s, added with 0.2 mL of dichloromethane for vortex shaking for 30 s, and added with 0.1 mL dd H₂O for vortex shaking for 30 s. Then the mixture solution was centrifuged at 9000 g for 1 min, and the upper layer was removed to obtain the organic dichloromethane layer. Then 0.3 mL methanol was added for vortex shaking for 30 s, and centrifuged at 9000 g for 2 min, and the supernatant was carefully removed to obtain a white precipitate. The residual solvent was dried with N₂, and add 200 μL of HEPES (20 mM HEPES,140 mM NaCl, 2% SDS) to solubilize the protein. The anti-CD47 protein ligation of complex III was determined to be 95% according to the test method of BCA kit.

### Example 6 Preparation of Anti-PD-L1/Anti-CD47-Targeted cGAMP/ENPP1-YZ2 Loaded Liposomes

450 mg of HSPC, 150 mg of cholesterol CHOL, 120 mg of DPPE-PEG₂₀₀₀60 mg of DPPE-PEG₂₀₀₀-MAL and 5 mg of ENPP1-YZ2 were added into 50 mL of dichloromethane, and transferred to a 1 L eggplant-shaped flask for vacuum spinning dry to form a film. Then, continue to spinning dry for more than 3 h to remove the residual trace organic solvent dichloromethane. 60 mL of 250 mM ammonium sulfate solution was added for hydration at 60° C. The hydrated liposome was extruded from a liposome extruder and filtered through a 100 nm polycarbonate microporous filtration membrane to form a homogeneous unicellular liposome. The homogeneous liposome was loaded into a 3500 Da dialysis bag and fully dialyzed to remove the free small-molecule inhibitor ENPP1-YZ2 and ammonium sulfate, where the buffer for dialysis was 500 mL of 5% dextrose solution; the dialysis was carried out three times; and the dialysate was changed every 6 h. 55 mg of cGAMP was added to the liposome solution and incubated under slight stirring with a magnetic stirrer in an oil bath at 70° C. for 2 h. After the incubation, the anti-PD-L1 VHH and anti-CD47 VHH prepared in Example 3 that had been sulfhydrylated (total weight of phospholipid:weight of anti-PD-L1 VHH: weight of anti-CD47 VHH=1 mg:20 μg: 20 μg) and the reducing agent (sodium hydrosulfite solution, with a final concentration of 1 mM) were added for incubation overnight at the room temperature in the dark. After that, the liposome loaded with the anti-PD-L1 VHH and the anti-CD47 VHH was loaded into a 30 kD dialysis bag and fully dialyzed to remove free anti-PD-L1 VHH, anti-CD47 VHH and cGAMP, where the buffer for dialysis was 500 mL of 5% dextrose solution; the dialysis was carried out three times; and the dialysate was changed every 6 h. The dialyzed liposome was added with 10 wt. % alginate and freeze-dried to obtain complex IV lyophilized powder, which was stored at −20° C.

The encapsulation rate determination of the complex IV was performed as follows. A certain amount of the complex II was added with 700 μL of emulsion breaker (methanol:isopropanol=7:3, V/V) and 300 μL of water, oscillated for uniform mixing, and centrifuged at 10,000 rpm for 1 min to obtain a supernatant. The supernatant was subjected to HPLC tests to determine the concentration of the drug through the concentration-peak area standard curve, and the encapsulation rate of the ENPP1-YZ2 was calculated to be 95%, and the encapsulation rate of cGAMP was calculated to be 94%.

The protein ligation of complex IV was determined as follows. 300 μL of a liposome solution was added with 0.4 mL of methanol for vortex shaking for 30 s, added with 0.2 mL of dichloromethane for vortex shaking for 30 s, and added with 0.1 mL dd H₂O for vortex shaking for 30 s. Then the mixture solution was centrifuged at 9000 g for 1 min, and the upper layer was removed to obtain the organic dichloromethane layer. Then 0.3 mL methanol was added for vortex shaking for 30 s, and centrifuged at 9000 g for 2 min, and the supernatant was carefully removed to obtain a white precipitate. The residual solvent was dried with N₂, and add 200 μL of HEPES (20 mM HEPES,140 mM NaCl, 2% SDS) to solubilize the protein. The anti-CD47 protein ligation of complex IV was determined to be 95% according to the test method of BCA kit.

### Example 7 Preparation of Folic Acid (FA)/Anti-PD-L1/Anti-CD47-Targeted cGAMP/ENPP1-YZ1 Loaded Liposomes

450 mg of HSPC, 150 mg of cholesterol CHOL, 120 mg of DPPE-PEG₂₀₀₀30 mg DSPE-PEG₂₀₀₀-FA, 60 mg of DPPE-PEG₂₀₀₀-MAL and 5 mg of ENPP1-YZ1 were added into 50 mL of dichloromethane, and transferred to a 1 L eggplant-shaped flask for vacuum spinning dry to form a film. Then, continue to spinning dry for more than 3 h to remove the residual trace organic solvent dichloromethane. 60 mL of 250 mM ammonium sulfate solution was added for hydration at 60° C. The hydrated liposome was extruded from a liposome extruder and filtered through a 100 nm polycarbonate microporous filtration membrane to form a homogeneous unicellular liposome. The homogeneous liposome was loaded into a 3500 Da dialysis bag and fully dialyzed to remove the free small-molecule inhibitor ENPP1-YZ1 and ammonium sulfate, where the buffer for dialysis was 500 mL of 5% dextrose solution; the dialysis was carried out three times; and the dialysate was changed every 6 h. 55 mg of cGAMP was added to the liposome solution and incubated under slight stirring with a magnetic stirrer in an oil bath at 70° C. for 2 h. After the incubation, the anti-PD-L1 VHH and anti-CD47 VHH prepared in Example 3 that had been sulfhydrylated (total weight of phospholipid:weight of anti-PD-L1 VHH:weight of anti-CD47 VHH=1 mg:20 μg:20 μg) and the reducing agent (sodium hydrosulfite solution, with a final concentration of 1 mM) were added for incubation overnight at the room temperature in the dark. After that, the liposome loaded with the anti-PD-L1 VHH and the anti-CD47 VHH was loaded into a 30 kD dialysis bag and fully dialyzed to remove free anti-PD-L1 VHH, anti-CD47 VHH and cGAMP, where the buffer for dialysis was 500 mL of 5% dextrose solution; the dialysis was carried out three times; and the dialysate was changed every 6 h. The dialyzed liposome was added with 10 wt. % alginate and freeze-dried to obtain complex V lyophilized powder, which was stored at −20° C.

The encapsulation rate determination of the complex V was performed as follows. A certain amount of the complex V was added with 700 μL of emulsion breaker (methanol:isopropanol=7:3, V/V) and 300 μL of water, oscillated for uniform mixing, and centrifuged at 10,000 rpm for 1 min to obtain a supernatant. The supernatant was subjected to HPLC tests to determine the concentration of the drug through the concentration-peak area standard curve, and the encapsulation rate of the ENPP1-YZ1 was calculated to be 94%, and the encapsulation rate of cGAMP was calculated to be 95%.

The protein ligation of complex V was determined to be 95%.

### Example 8 Study on the Inhibitory Effect of Multi-Targeted Complex Drug Against Solid Tumors

### 1. Experimental Animals and Feeding Conditions

Specie and strain: BalB/C ordinary mice and C57BL/6 ordinary mice.

Sex: male.

Weighing: 20-22 g.

Age: 7-8 weeks old.

Grade: SPF.

Source: Shanghai Slaughter Laboratory Animal Co.

All mice were fed and watered freely and kept at room temperature (23±2° C.) The feed and water were autoclaved, and all experimental feeding processes were SPF grade.

### 2. Drug Doses

Mice were injected intraperitoneally with multi-targeted complex drug against tumors, and the dosages were shown in Table 1.

TABLE 1
Dosages of drugs

| | | | Anti-PD- | |
|---|---|---|---|---|
| | cGAMP | ENPP1 | L1 VHH | Anti-CD47 |
| | (mg/kg/ | inhibitor | (mg/kg/ | VHH |
| Drugs | day) | (mg/kg/day) | day) | (mg/kg/day) |
| Complex I | 10 | 5 (ENPP1-YZ1) | — | — |
| Complex II | 10 | 5 (ENPP1-YZ1) | — | 2.5 |
| Complex III | 10 | 5 (ENPP1-YZ1) | 2.5 | 2.5 |
| Complex IV | 10 | 5 (ENPP1-YZ2) | 2.5 | 2.5 |
| Complex V | 10 | 5 (ENPP1-YZ1) | 2.5 | 2.5 |
| cGAMP | 10 | — | — | — |
| ENPP1-YZ1 | — | 5 | — | — |
| ENPP1-YZ2 | — | 5 | — | — |
| Anti-PD-L1 VHH | — | — | 2.5 | — |
| Anti-CD47 VHH | — | — | — | 2.5 |

### 3. Control Tests.

Negative control: PBS solution.

Positive control: cGAMP with a dose of 10 mg/kg.

### 4. Administration Method

Route of administration: intraperitoneal injection.

Dose of the novel multi-target immune complex: 200 μL/each;

Dosing frequency: once a day for 21 consecutive days;

Number of animals per group: 10.

### 5. Tumor Cell Lines

Used herein are mouse colorectal cancer cell line CT26, mouse breast cancer cell line 4T1 and mouse melanoma cell line B16F10, which were purchased from the cell bank of Chinese Academy of Sciences.

### 6. Main Steps of the Experiment

Establishment and intervention of tumor-model mice

Cancer cells were cultured, passaged, collected at the logarithmic phase of cells, and prepared into a cell suspension at a concentration of (1.0×10⁷) per ml. Mice were injected with 0.2 ml of the cell suspension at the axilla of the right forelimb (cell number 2.0×10⁶ cells/each), and tumorigenicity was successful in about 8 days.

These mice were randomly divided into 11 groups equally, namely, group A: negative control group (PBS group), group B: positive control (cGAMP) group (dose: 10 mg/kg), group C: ENPP1-YZ1 group (dose: 5 mg/kg), group D: ENPP1-YZ2 group (dose: 5 mg/kg), group E: anti-PD-L1 VHH group (dose: 2.5 mg/kg), group F: anti-CD47 VHH group (dose: 2.5 mg/kg), group G: complex I group, group H: complex II group, group I: complex III group, group J: complex IV group, and group K: complex V group. The mice were administered once a day for 21 days. After 21 days, the mice were executed, the tumor weight was weighed, and the tumor inhibition rate was calculated by [1-average tumor weight of experimental groups (groups B, C, D, E, F, G, H, I, J, and K)/average tumor weight of group A]×100%.

The mouse colorectal cancer cell line CT26 was prepared and injected into BalB/C ordinary mice. The mouse breast cancer cell line 4T1 was prepared and injected into BalB/C ordinary mice. The melanoma cell line B16F0 was prepared and injected into C57BL/6 ordinary mice. The anti-tumor effects of different drugs were observed.

### Statistical Analysis

Data were expressed as x±s and processed using a SPSS 10.0 software. One-way ANOVA test was used to compare the significance of the differences in tumor weight between groups, with a significance level a=0.05.

### Experimental Results

Subcutaneous transplantation tumor models were successfully prepared after subcutaneous inoculation of tumor cells in mice, and the novel multi-target immune complexes all significantly inhibited tumor growth. The tumor weights of the experimental groups were all significantly lower than those of the negative control group after 21 days of administration of the drugs (P<0.05, P<0.01). The effects of the novel multi-target immune complexes were all superior to the individual anti-tumor drugs including cGAMP, ENPP1 inhibitor, and nanoantibodies. It indicated that the novel multi-targeted immune complexes had significantly improved anti-tumor effects. The anti-CD47/anti-PD-L1 VHH immune-targeting complexes had anti-tumor efficacy significantly better than single antibody (anti-CD47). The efficacy of the anti-CD47/anti-PD-L1 VHH multi-targeting complex containing folic acid was significantly improved, which had the best efficacy. The specific results were shown in Tables 2-4.

In addition, the novel multi-targeted immune complexes showed a significant inhibitory effect on lung metastasis of breast cancer cells in mice. Dissections were performed on mice that had been executed for the 4T1 breast cancer study, where the lung tissues were carefully peeled off and photographed for observation after fixation with 4% paraformaldehyde. It could be clearly seen that the lungs of the mice in the model group were black and existed multiple white tumor nodules with various sizes on the surface. The lungs of mice in the STING agonist cGAMP group and the ENPP1 inhibitor group showed a decrease in the number of tumor nodules compared with that in the model group, and the lungs of mice in the anti-PD-L1 VHH group and the anti-CD47 VHH group also showed a small decrease in the number of tumor nodules compared with that in the model group. It showed that the multi-targeted complex administration groups had better inhibition effect on the lung metastasis of breast cancer cells, where the mice had a normal lung tissue volume and a small number of tumor nodules on the surface. By statistical analysis of the tumor nodules in the lung tissues of mice, the results of the efficacy of inhibiting the lung metastasis of breast cancer cells were shown in Table 3.

TABLE 2
Inhibition effects of novel multi-targeted complexes
on subcutaneous graft tumors of murine colorectal
cancer cell CT26 in BalB/C mice (n = 10, mean ± SD)

| | | Average tumor |
|---|---|---|
| | | suppression |
| Groups | Average tumor weight (g) | rate (%) |
| Negative control group | 2.265 ± 0.237 (g) | — |
| Positive control (cGAMP) | 0.706 ± 0.152 (g)** | 68.8 |
| group | | |
| ENPP1-YZ1 group | 0.816 ± 0.203 (g)** | 63.9 |
| ENPP1-YZ2 group | 0.805 ± 0.218 (g)** | 64.5 |
| Anti-PD-L1 VHH group | 1.735 ± 0.206 (g)** | 23.4 |
| Anti-CD47 VHH group | 1.807 ± 0.187 (g)** | 20.2 |
| Complex I Group | 0.455 ± 0.176 (g)** | 79.9 |
| Complex II Group | 0.364 ± 0.135 (g)** | 83.9 |
| Complex IIIGroup | 0.216 ± 0.161 (g)** | 90.5 |
| Complex IV Group | 0.188 ± 0.182 (g)** | 91.7 |
| Complex V Group | 0.125 ± 0.102 (g)** | 94.5 |

Noted:
*P < 0.05 vs negative control group; and
**P < 0.01 vs negative control group.

TABLE 3
Inhibition effects of novel multi-targeted complexes
on subcutaneous graft tumors of mouse breast cancer
cell line 4T1 in BalB/C mice (n = 10, mean ± SD)

| | | Average | Number of |
|---|---|---|---|
| | | tumor | metastatic |
| | Average tumor | suppression | lung |
| Groups | weight (g) | rate (%) | nodules |
| Negative control group | 2.275 ± 0.229 (g) | — | 35 |
| Positive control | 0.775 ± 0.221 (g)** | 65.9 | 12 |
| (cGAMP) group | | | |
| ENPP1-YZ1 group | 0.898 ± 0.136 (g)** | 60.5 | 21 |
| ENPP1-YZ2 group | 0.852 ± 0.223 (g)** | 62.5 | 20 |
| Anti-PD-L1 VHH group | 1.752 ± 0.214 (g)** | 22.9 | 27 |
| Anti-CD47 VHH group | 1.805 ± 0.176 (g)** | 20.2 | 25 |
| complex I group | 0.445 ± 0.176 (g)** | 80.4 | 10 |
| complex II group | 0.382 ± 0.156 (g)** | 83.2 | 8 |
| complex III group | 0.218 ± 0.165 (g)** | 90.4 | 5 |
| complex IV group | 0.179 ± 0.129 (g)** | 92.1 | 4 |
| complex V group | 0.109 ± 0.103 (g)** | 95.2 | 2 |

Noted:
*P < 0.05 vs negative control group; and
**P < 0.01 vs negative control group.

TABLE 4
Inhibition effects of novel multi-targeted complexes
on subcutaneous graft tumors of mouse melanoma cell
line B16F0 in C57BL/6 mice (n = 10, mean ± SD)

| | | Average tumor |
|---|---|---|
| | Average tumor | suppression |
| Groups | weight (g) | rate (%) |
| Negative control group | 2.386 ± 0.216 (g) | |
| Positive control (cGAMP) group | 0.808 ± 0.137 (g)** | 66.1 |
| ENPP1-YZ1 group | 0.943 ± 0.208 (g)** | 60.5 |
| ENPP1-YZ2 group | 0.926 ± 0.227 (g)** | 61.2 |
| Anti-PD-L1 VHH group | 1.758 ± 0.212 (g)** | 26.3 |
| Anti-CD47 VHH group | 1.809 ± 0.217 (g)** | 24.2 |
| complex I group | 0.516 ± 0.215 (g)** | 78.4 |
| complex II group | 0.406 ± 0.231 (g)** | 82.9 |
| complex III group | 0.205 ± 0.118 (g)** | 91.4 |
| complex IV group | 0.181 ± 0.123 (g)** | 92.4 |
| complex V group | 0.119 ± 0.102 (g)** | 95.0 |

Noted:
*P < 0.05 vs negative control group; and
**P < 0.01 vs negative control group.

### Example 9 Study on the Effects of the Ratio of Active Components of Multiple-Targeted Anti-Tumor Complexes on Anti-Tumor Efficacy and Safety

Experimental animals and feeding conditions thereof

Specie and strain: BalB/C ordinary mice and C57BL/6 ordinary mice.

Sex: male.

Weighing: 20-22 g.

Age: 7-8 weeks old.

Grade: SPF.

Source: Shanghai Slac Laboratory Animal Co., Ltd.

All mice were fed and watered freely, and kept at room temperature (23±2° C.) The feed and water were autoclaved, and all experimental feeding processes were SPF grade.

The multi-target complexes with different active components were as shown in Table 5 below.

TABLE 5
Multi-target complexes with different ratios of active components

| | cGAMP | ENPP1-YZ1 | Anti-PD-L1 | Anti-CD47 |
|---|---|---|---|---|
| Complexes | (mg) | inhibitor (mg) | VHH (mg) | VHH (mg) |
| I | 10 | 10 | 100 | 100 |
| II | 10 | 10 | 50 | 50 |
| III | 10 | 5 | 25 | 25 |
| IV | 10 | 5 | 5 | 5 |
| V | 10 | 5 | 2.5 | 2.5 |
| VI | 10 | 2.5 | 40 | 40 |
| VII | 10 | 20 | 2.5 | 2.5 |
| VIII | 20 | 5 | 40 | 40 |

In the preparation of the multi-targeted complexes for immune-targeted anti-tumor, the effect of the ratio of active compositions of the multi-targeted complex on the efficacy and safety was firstly studied, including weight, survival rate and tumor suppression rate of the mice. The mice were intraperitoneally injected with the multi-targeted complexes with different ratio of effective components, and the contents of the effective components in the complexes administered to the mice were shown in Table 5 in kilogram of mice per daily.

### Control Tests

Negative control: PBS solution.

### Administration Method

Route of administration: intraperitoneal injection.

Dose of the multi-target complex: 200 μl/each;

Dosing frequency: 1 time per day for 21 consecutive days;

Number of animals per group: 10.

### Tumor Cell Lines

Used herein is mouse colorectal cancer cell line CT26, which was purchased from the cell bank of Chinese Academy of Sciences.

The establishment of tumor-model mice was referred to Example 8.

### Test Results

Subcutaneous graft tumor models were successfully prepared after subcutaneous inoculation of mice with tumor cells. After 21 days of administration of multi-target immune complexes with various compositional ratios, the results were shown in Table 6.

TABLE 6
Effects of active compositional ratios of the multi-
targeted complexes on survival rate and tumor suppression
rate, and weight increase rate of mice

| | Survival | Tumor inhibition | Weight change |
|---|---|---|---|
| Complex | rate (%) | rate (%) | rate (100%) |
| I | 0 | — | — |
| II | 60 | 95 | −25% |
| III | 100 | 93 | −15% |
| IV | 100 | 92 | −8% |
| V | 100 | 90 | −2% |
| VI | 100 | 91 | −6% |
| VII | 0 | — | — |
| VIII | 80 | 93 | −16% |
| Tumor model group | 100 | — | −8% |
| Normal mice group | 100 | — | Control group |

It could be seen that the ratio of the active components in the complexes and its administration dose are importantly related to the anti-tumor efficacy and safety. Hence, it was critical to regulate the appropriate ratio. The results showed that when the dose of the STING agonist was 10 mg/kg/day, the effective and safe dose for the ENPP1 inhibitor was 2.5-5 mg/kg/day, and for the immune checkpoint inhibitor (nanoantibodies) was 2.5-50 mg/kg/day. When the dose of the STING agonist was 10 mg/kg/day, the effective and safe dose for the ENPP1 inhibitor was greater than or equal to 10 mg/kg/day, and for the immune checkpoint inhibitor (nanoantibodies) was greater than or equal to 100 mg/kg/day, the mice experienced serious safety issues, such as death. The analysis results on the cause of death in mice showed that the combination of the STING agonist, the ENPP1 inhibitor and the immune checkpoint inhibitor were too strong, posing an immune storm in mice, thereby leading to death. Excessive use of the STING agonist alone did not generate an immune storm because there was ENPP1 in the body that could degrade excessive endogenous STING agonist cGAMP (non-endogenous STING agonists could not be degraded by ENPP1). If the STING agonist and the ENPP1 inhibitor were excessively used at the same time, an immune storm was generated. The immune checkpoint inhibitor antibodies played two roles in the multi-targeting complexes: blocking immune escape and targeting function (targeting to tumor cells/or immune cells), both of which could enhance anti-tumor efficacy.

What is claimed is:

1. A liposome carrier-based multi-target complex, comprising:
a stimulator of interferon genes (STING) agonist;
an immune checkpoint inhibitor;
an ectonucleotide pyrophosphatase/phosphodiesterase (ENPP1) inhibitor; and
a liposome;
wherein the liposome is a closed unilamellar vesicle having a concentric lipid bilayer membrane and a hydrophilic core; the STING agonist is located in the hydrophilic core, and the ENPP1 inhibitor is located in a hydrophobic interlayer of the concentric lipid bilayer membrane; and the immune checkpoint inhibitor is linked to an outer surface of the liposome;
the immune checkpoint inhibitor is configured to target at least two different immune checkpoint or antigenic epitopes; and
a weight ratio of the STING agonist to the ENPP1 inhibitor is 10:2.5-10, excluding 10:10; and a weight ratio of the STING agonist to the immune checkpoint inhibitor is 10:2.5-100, excluding 10:100.

2. The liposome carrier-based multi-target complex of claim 1, wherein the weight ratio of the STING agonist to the ENPP1 inhibitor is 10:2.5-5; the weight ratio of the STING agonist to the immune checkpoint inhibitor is 10:5-80; the STING agonist is an agonist of a cyclic GMP-AMP synthetase (cGAS)-STING-cyclic GMP-AMP (cGAMP)-interferon regulatory factor 3 (IRF3) immune pathway; and the immune checkpoint inhibitor is an antibody against an immune checkpoint or an antigen-binding fragment thereof.

3. The liposome carrier-based multi-target complex of claim 2, wherein the weight ratio of the STING agonist to the immune checkpoint inhibitor is 10:50 or 10:10;
the agonist is selected from the group consisting of cyclic dinucleotides and metal compounds thereof, aminobenzimidazoles, xanthones, acridones, benzothiophenes, benzodioxoles and a combination thereof;
the immune checkpoint is selected from the group consisting of programmed cell death protein 1 (PD-1)/programmed cell death ligand (PD-L1), CD47, vascular endothelial growth factor (VEGF), poliovirus receptor-related immunoglobulin domain-containing protein (PVRIG), T cell immunoreceptor with immunoglobulin and ITIM domain (TIGIT), NKG2A, leukocyte immunoglobulin like receptor B4 (LILRB4), LILRB2, lymphocyte activation gene 3 (LAG-3), OX40, cytotoxic T lymphocyte antigen 4 (CTLA-4), T-cell immunoglobulin and mucin domain-containing protein 3 (TIM-3), V-domain immunoglobulin suppressor of T cell activation (VISTA), immunoglobulin-like domain containing receptor 2 (ILDR2), killer cell immunoglobulin-like receptor (KTR), major histocompatibility class II (MHCII), glucocorticoid-induced tumor necrosis factor receptor-related protein (GITR), 4-1BB and a combination thereof, and the immune checkpoint inhibitor is the antibody against the immune checkpoint.

4. The liposome carrier-based multi-target complex of claim 3, wherein the agonist is a cyclic dinucleotide or a cyclic dinucleotide metal compound; the immune checkpoint is PD-1/PD-L1 or CD47; and the antibody is a nanobody (VHH).

5. The liposome carrier-based multi-target complex of claim 4, wherein the agonist is 2′3′-cGAMP.

6. The liposome carrier-based multi-target complex of claim 1, wherein the ENPP1 inhibitor is a 4(3H)-quinazolinone derivative; and
raw materials of the liposome comprise a phospholipid and cholesterol; the phospholipid is selected from hydrogenated soybean phospholipid, dipalmitoyl phosphatidylcholine, dipalmitoyl phosphatidylglycerol, dipalmitoyl-phosphatidylethanolamine-polyethylene glycol 2000, dipalmitoyl phosphatidylethanolamine-polyethylene glycol 2000-maleoethanolamine, 1,2-distearoyl-SN-glycerol-3-phosphoethanolamine-N-maleimide-polyethylene glycol 2000 (DSPE-PEG2000-MAL) and a combination thereof, and a weight ratio of the phospholipid to the cholesterol is 18-25:5.

7. The liposome carrier-based multi-target complex of claim 6, wherein the ENPP1 inhibitor is selected from the group consisting of 2-[(4-methyl-(thiazolo[4,5-B]pyridinyl)-2-thio)methyl]-7-fluoro-quinazoline-4(3H)-one, 2-[(5-methoxy-(imidazo[4,5-B]pyridinyl)-2-thio)methyl]-7-fluoro-quinazoline-4(3H)-one and 2-[(5-chloro-(imidazo[4,5-B]pyridine)-2-thio)methyl]-7-methoxycarbonyl-quinazoline-4(3H)-one; and
the weight ratio of the phospholipid to the cholesterol is 19-21:5.

8. The liposome carrier-based multi-target complex of claim 1, further comprising:
a tumor-targeting reagent;
wherein the tumor-targeting reagent is bridged to the outer surface of the liposome through a chemical bond; and
the tumor-targeting reagent is a ligand targeting a receptor specifically expressed by a tumor cell.

9. The liposome carrier-based multi-target complex of claim 8, wherein the tumor-targeting reagent is folic acid.

10. The liposome carrier-based multi-target complex of claim 1, wherein the liposome carrier-based multi-target complex is prepared through steps of:
(S1) subjecting raw materials of the liposome and the ENPP1 inhibitor to thin film hydration to prepare a loaded liposome, wherein the raw materials of the liposome comprise a phospholipid and cholesterol; and
(S2) subjecting the loaded liposome and the STING agonist to a first incubation under stirring, and adding the immune checkpoint inhibitor for a second incubation under stirring to obtain the liposome carrier-based multi-target complex;
wherein the first incubation is performed at 65-75° C. for 1.5-2.5 h, and the second incubation is performed at 20-25° C. overnight in the dark.

11. The liposome carrier-based multi-target complex of claim 10, wherein the thin film hydration is performed in the presence of a tumor-targeting reagent; and
a weight ratio of the STING agonist to the tumor-targeting reagent is 10:2.5-10, excluding 10:10.

12. A drug delivery platform, comprising:
the liposome carrier-based multi-target complex of claim 1.

13. A pharmaceutical composition, comprising:
the liposome carrier-based multi-target complex of claim 1; and
a pharmaceutically-acceptable carrier;
wherein an administration route of the pharmaceutical composition comprises injection, drip and oral administration;
the injection is intravenous injection, intramuscular injection or subcutaneous injection; and
the drip is intravenous drip or nasal drip.

14. The pharmaceutical composition of claim 13, further comprising:
a therapeutic agent;
wherein the therapeutic agent comprises an antibody against a coronavirus spike protein, or fragments of the antibody.

15. The pharmaceutical composition of claim 13, further comprising:
a therapeutic agent;
wherein the therapeutic agent comprises a peptide or an antibody targeting a receptor protein of a blood-brain barrier, or fragments of the peptide or antibody.

16. A method for treating a tumor or a tumor metastasis in a subject in need thereof, comprising:
administering a therapeutically effective amount of the pharmaceutical composition of claim 13 to the subject;
wherein the tumor is a solid tumor; the solid tumor is selected from the group consisting of colorectal cancer, breast cancer, ovarian cancer, prostate cancer, pancreatic cancer, testicular cancer, lung cancer, nasopharyngeal cancer, esophageal cancer, renal cancer, glioma, melanoma, malignant lymphoma, head and neck cancer, thyroid cancer and osteogenic sarcoma; and the tumor metastasis is selected from the group consisting of lung metastasis, liver metastasis, lymphatic metastasis, brain metastasis and bone metastasis.

17. A method for treating a viral inflammation associated with a coronavirus in a subject in need thereof, comprising:
administering a therapeutically effective amount of the pharmaceutical composition of claim 14 to the subject;
wherein the coronavirus is selected from the group consisting of 2019-novel coronavirus (2019-nCoV), influenza virus and human immunodeficiency virus (HIV); and the viral inflammation is selected from the group consisting of Corona Virus Disease 2019 (COVID-19), viral nephritis, viral encephalitis, viral enteritis and viral hepatitis.

18. A method for treating a neurodegenerative disease in a subject in need thereof, comprising:
administering a therapeutically effective amount of the pharmaceutical composition of claim 15 to the subject;
wherein the neurodegenerative disease is selected from the group consisting of Alzheimer's disease, Parkinson's disease, amyotrophic lateral sclerosis, multiple sclerosis, ataxia telangiectasia, bovine spongiform encephalopathy, Creutzfeldt-Jakob disease, Huntington's chorea, spinocerebellar atrophy, spinal muscular atrophy, spastic paraplegia and myasthenia gravis.

19. A method for treating a brain disease in a subject in need thereof, comprising:
administering a therapeutically effective amount of the pharmaceutical composition of claim 15 to the subject;
wherein the brain disease is selected from the group consisting of ischemic cerebrovascular injury, craniocerebral injury, encephalitis, and brain tumor.


SEQUENCE LISTING (2 sequences)

SEQ ID NO: 1 - protein, 116 aa, synthetic construct
  source 1..116: note = Aritifical sequence
QVQLQESGGGLVQTGGSLRLSCAASGSTVSSSMMAWWRQTPGNQRELVALVASGNNTNYVDSVKGRFTVSRDNAKNTMYLQMNSLKPEDTAVYYCRILSVNGIWYWGQGTQVTVSS

SEQ ID NO: 2 - protein, 121 aa, synthetic construct
  source 1..121: note = Artificial sequence
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSS